(19) United States
(12) Patent Application Publication
(10) Pub. No.: US 2019/0335976 A1
(43) Pub. Date: Nov. 7, 2019

(54) AUTOMATED THRESHOLD SANITIZING APPARATUS

(71) Applicant: Elijah Valenzuela, Camarillo, NY (US)

(72) Inventor: Elijah Valenzuela, Camarillo, NY (US)

(21) Appl. No.: 15/968,406

(22) Filed: May 1, 2018

(51) Int. Cl. A47L 23/26; A61L 2/24; A61L 2/18

(52) U.S. Cl. CPC A47L 23/263; A61L 2/24; A61L 2202/15; A61L 2202/14; A61L 2202/17; A61L 2/18

(57) ABSTRACT

An automated threshold sanitizing apparatus cleans and sanitizes the soles of footwear and feet in a hands-free manner. The apparatus dispenses a pre-saturated sanitized sheet across the threshold for the user to clean and sanitize the soles. A receiving roller draws the sanitized sheet from a dispensing roller to advance a predetermined section of the sheet across the threshold. The receiving and dispensing rollers are housed in a cartridge on opposite sides of threshold. A contaminated section of the sheet is rolled onto the receiving roller for disposal, and replaced by a new sheet that is dispensed across the threshold. A sensor detects motion across threshold, such as footwear moving away from threshold, triggering transmission of a signal to a motor. The motor drives a gear roller that meshes with, and rotates, the receiving roller to draw in sheet. The cartridges include a lid for accessing and replacing rollers.

### FIELD OF THE INVENTION

The present invention relates generally to an automated threshold sanitizing apparatus. More so, the present invention relates to a sanitizing apparatus that cleans and sanitizes the soles of footwear and feet in a hands-free manner by dispensing a pre-saturated sanitized sheet across the threshold for the user to clean and sanitize the soles; whereby a receiving roller draws the sanitized sheet from a dispensing roller to advance a predetermined section of the sheet across the threshold; whereby the receiving and dispensing rollers are housed in a cartridge on opposite sides of the threshold, and that enable replacement of respective rollers; whereby a sensor detects motion across the threshold, such as footwear moving away from the sheet, and transmits a signal to a motor, so that the motor drives a gear roller that meshes with, and rotates, the receiving roller; and whereby a contaminated section of the sheet is rolled onto the receiving roller for disposal, and replaced by a new sheet that is dispensed across the threshold.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Typically, footwear, such as footwear, sandals, socks, etc., and even feet are carriers of multiple contamination agents that are often introduced into interiors of homes, hospitals, schools, and offices from various sources of contamination. Although any portion of the footwear is known to carry contamination, due to contact with contamination from surfaces, the majority of contamination is carried on the soles of footwear and bare feet.

Often, people acquire a great deal of dirt on the soles of their footwear. Surfaces such as roads and sidewalks have a high content of food waste, animal excrement, organic decompositions, dirt and debris. This dirt contains harmful bacteria that could pose serious health risks if brought into a household or dwelling. If dirt is not removed prior to entry, it can be tracked into a building and deposited on clean floors. Dirt left on floors results in an unclean appearance and promotes the proliferation of harmful bacteria.

It is known that doormats can reduce the risk of unclean floors and bacterial growth by providing a surface that may be rubbed upon to affect the removal of dirt. Entry mats are commonly placed outside the doorways of homes, office buildings and businesses to remove dirt and debris from the soles of footwear prior to an individual's entry into the building. Unfortunately, these mats are typically simple, carpeted or moisture-absorbing surfaces that collect particulate dirt and debris. After continual use, these mats become imbedded with dirt, dust and debris that limit their usefulness when cleaning a user's feet or footwear.

Further, a difficulty, however, with most mats is that they are directed toward a single function, that is, the mat is generally a normal wiping mat that enables the user to simply apply the shoe to the mat with a rubbing or scuffing motion to the shoe to wipe the dirt and other material from the shoe. With such single function mats, the shoe does not get particularly clean and there is no way to include some disinfecting function to rid the shoe of potentially harmful materials that can then be spread to other areas, such as the inside of the home or automobile of the person.

A quick wipe on a doormat will only clean so much and has no disinfecting capabilities, leaving the floors and carpets to absorb what is left on the soles of the footwear or from the bare feet. The door mat itself can become a central point in actually infecting visitors since numerous visitors actually wipe their feet and leave germs, viruses and the like on the mat for the next person to encounter.

Typically, vacuuming the carpets where children often play cannot adequately remove all the bacteria, germs, and dirt which the mat misses and which can filter deep into the floor coverings. Because of this lack of hygiene on entry to homes and businesses, to be fully protected floors must be mopped with a disinfectant and carpets steam cleaned often to kill or remove contaminants missed at the door. Furthermore, in medical facilities shoe coverings are worn in operating rooms, but in moving around the hospital from room to room medical personnel have the potential of carrying infectious materials from one place to another on their shoes.

Often, contaminated footwear is a major concern, especially in hospital settings. Often, hospitals have isolation wards for people that have highly contagious diseases such as necrotizing fasciitis and Methicillin-resistant Staphylococcus aureus (MRSA). The hospitals attempt to control the spread of such diseases by maintaining a negative air pressure in these wards (so air flows in when a door is opened), constant filtration of the air in the wards, constant germicidal treatment, wearing of disposable outer garments, etc.

It is also known that workers in agricultural fields are constantly walking in fertilizers and insecticides, which cling to their feet and can impregnate their footwear. Working with animals creates additional problems where individuals are required to walk through fresh feces that can cling to their footwear.

Other proposals have involved cleaning the soles of feet and footwear. The problem with these cleaning methods and systems is that they do not both clean and sanitize the soles of the feet or footwear. Also, the sanitizing sheet or pad is not positioned for cleaning the soles of the footwear or feet in a hands-free manner. Even though the above cited footwear cleaning methods and systems meet some of the needs of the market, an automated threshold sanitizing apparatus. More so, the present invention relates to a sanitizing apparatus that cleans and sanitizes the soles of footwear and feet in a hands-free manner by dispensing a pre-saturated sanitized sheet across the threshold for the user to clean and sanitize the soles is still desired.

### SUMMARY

Illustrative embodiments of the disclosure are generally directed to an automated threshold sanitizing apparatus. The sanitizing apparatus serves to clean and sanitize the soles of footwear and feet in a hands-free manner, and prior to a user's entry across a threshold. The sanitizing apparatus dispenses a pre-saturated sanitized sheet across the threshold for the user to clean the soles of the footwear and feet prior to entering a building. A dispensing roller and a receiving roller advance a predetermined section of the sheet across the threshold, such that a contaminated section of the sheet is rolled onto the receiving roller for disposal, and replaced by a new sheet from the dispensing roller. Both rollers are housed in a cartridge that includes a lid for enabling access to replace and discard the rollers.

The pre-saturated sanitized sheet is maintained in a taut configuration between the rollers to prevent lateral movement by the sheet while the footwear and feet are wiping. After the user cleans and sanitizes the soles of the footwear or feet, and crosses the threshold, a sensor detects absence of the user. The sensor transmits a signal based upon the motion or presence of the object, such as the footwear moving away from the threshold.

The signal actuates the motor for a predetermined duration that is sufficient to advance a fresh section of the sheet onto the threshold. The motor drives a gear roller having teeth that mesh with the outer surface of the receiving roller. This causes the receiving roller to draw the sheet across the threshold until a new section of the sheet is made available for the subsequent users to similarly clean and sanitize the soles of the footwear and feet. The contaminated section of the sheet rolls onto the receiving roller where the receiver roller and contaminated sheets are subsequently removed for disposal.

In another aspect, the receiving cartridge and the dispensing cartridge are disposed in a spaced-apart relationship separated by a threshold.

In yet another aspect, the threshold comprises a doormat.

In yet another aspect, the sanitizing formulation of the pre-saturated sanitized sheet comprises ethanol.

In yet another aspect, the dispensing cartridge is generally rectangular.

In yet another aspect, the dispensing cartridge is fabricated from at least one of the following: stainless steel, aluminum, metal, a rigid polymer, and wood.

In yet another aspect, the motor comprises an electrical motor.

In yet another aspect, the fastening mechanism of the receiving roller includes at least one of the following: a slot, a plurality of hooks, a clamp, and an adhesive.

In yet another aspect, the receiving cartridge is generally rectangular.

In yet another aspect, the receiving cartridge is fabricated from at least one of the following: stainless steel, aluminum, metal, a rigid polymer, and wood.

In yet another aspect, the apparatus further comprises a track extending between the dispensing and receiving cartridges.

In yet another aspect, the pre-saturated sanitized sheet advances parallel with the track.

In yet another aspect, the sensor is a motion sensor.

In yet another aspect, the signal is transmitted to the motor.

In yet another aspect, the object comprises footwear or feet.

In yet another aspect, the sensor transmits the signal based upon the footwear or feet moving away from between the dispensing and receiving cartridges.

In yet another aspect, the object comprises footwear or feet wipe across the section of pre-saturated sanitized sheet between the dispensing and receiving cartridges.

One objective of the present invention is to provide a hands-free system capable of cleaning and sanitizing the soles of footwear across a threshold.

Another objective is to provide an automated threshold sanitizing apparatus that sanitizes in addition to cleaning footwear and bare feet.

Another objective is to provide an automated threshold sanitizing apparatus for more effectively cleaning, sanitizing, refreshing, and deodorizing bare feet.

Another objective is to provide a disposable pre-saturated sanitized sheet for cleaning and sanitizing the soles of the footwear and feet.

Yet another objective is to maintain the pre-saturated sanitized sheet in a taught disposition between the rollers, so as to prevent lateral movement as a user wipes the feet.

Yet another objective is to provide access inside the dispensing cartridge to replace the finished roll of pre-saturated sanitized sheet; and access inside the receiving cartridge to discard the contaminated sheet.

Yet another objective is to provide a cleaning and sanitizing apparatus that can be placed anywhere in a residential or commercial structure where desired.

Yet another objective is to provide a cleaning and sanitizing apparatus that is visually attractive, portable, easy to maintain, and easy to use.

Other systems, devices, methods, features, and advantages will be or become apparent to one with skill in the art upon examination of the following drawings and detailed description. It is intended that all such additional systems, methods, features, and advantages be included within this description, be within the scope of the present disclosure, and be protected by the accompanying claims and drawings.

### BRIEF DESCRIPTION OF THE DRAWINGS

The invention will now be described, by way of example, with reference to the accompanying drawings, in which:

FIG. 1 illustrates a side perspective view of a user removing contaminants from the footwear with an exemplary automated threshold sanitizing apparatus, in accordance with an embodiment of the present invention;

FIG. 2 illustrates a top view of the automated threshold sanitizing apparatus, in accordance with an embodiment of the present invention;

FIG. 3 illustrates a sectioned side view of the automated threshold sanitizing apparatus, showing a sensor transmitting a signal to advance an exemplary pre-saturated sanitized sheet over the threshold, in accordance with an embodiment of the present invention; and

FIG. 4 illustrates a sectioned side view of the automated threshold sanitizing apparatus, shown in FIG. 3 with the lids for the cartridges open to enable access to the dispensing roller and receiving roller for replacement, in accordance with an embodiment of the present invention.

Like reference numerals refer to like parts throughout the various views of the drawings.

### DETAILED DESCRIPTION OF THE INVENTION

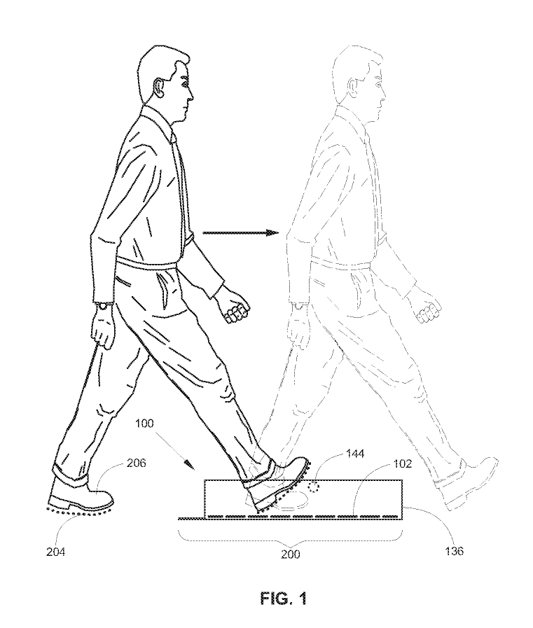

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Specific dimensions and other physical characteristics relating to the embodiments disclosed herein are therefore not to be considered as limiting, unless the claims expressly state otherwise.

An automated threshold sanitizing apparatus 100 is referenced in FIGS. 1-4. The automated threshold sanitizing apparatus 100, hereafter “apparatus 100”, provides hands-free means to clean and sanitize the soles of footwear 206 and feet over a threshold 200, prior to the footwear 206 or feet crossing the threshold 200. Apparatus 100 is unique in that it provides a disposable, pre-saturated sanitized sheet 102 that positions over the threshold 200 for the soles of the footwear 206 or feet to be cleaned and sanitized.

The easy disposability, sanitizing formulation, and automatic advancement and replacement of the pre-saturated sanitized sheet 102 across the threshold 200 helps address the issue of contaminants 204 on the footwear 206, such as germs, bacteria, pathogens, chemicals, toxins, and debris on the soles of the footwear 206 being tracked across a threshold 200 by sanitizing, disinfecting, and cleaning the soles of the footwear 206; and sanitizing, refreshing, cleaning, and deodorizing the soles of bare feet crossing threshold 200.

As FIG. 1 illustrates, apparatus 100 is configured to enable the hands-free cleaning and sanitizing of the soles of footwear 206 and feet prior to a user's entry across a threshold 200. The threshold 200 may include, without limitation, a doormat, a doorway, an entryway to a building, vehicle, or locale. The footwear 206 may include, without limitation, a shoe, a sandal, a slipper, and a boot.

Apparatus 100 dispenses a pre-saturated sanitized sheet 102 in incrementally new, clean sections across the threshold 200 for the user to clean the soles of the footwear 206 and feet prior to crossing the threshold 200. A dispensing roller 106 and a receiving roller 124 work together to advance a clean section of the sheet 102 across the threshold 200, such that a contaminated section of the sheet 102 is rolled off the threshold 200 and onto receiving roller 124 for disposal, and replaced by the new sheet from the dispensing roller 106. Both rollers 106, 124 are housed in a cartridge 116, 136 that includes a lid 118, 138 for enabling access to replace the spent dispensing roller 106 and discard the contaminated receiving roller 124.

Pre-saturated sanitized sheet 102 is maintained in a taut configuration between the rollers 06, 124 to prevent lateral movement by the sheet 102 while the footwear 206 and feet are wiping. After the user cleans and sanitizes the soles of the footwear 206 or feet, and crosses the threshold 200, a sensor 144 detects absence of the footwear 206 or feet. Sensor 144 then transmits a signal 146 based upon the motion, or presence of the object 202. Signal 146 actuates the motor 134 for a predetermined duration. Motor 134 drives a gear roller 130 having teeth 132 that mesh with the outer surface 108 of the receiving roller 124.

Consequently, this causes receiving roller 124 to draw the sheet 102 a predetermined distance across threshold 200, or until a clean section of the sheet is made available for the subsequent users to clean and sanitize the soles of the footwear 206 and feet. The contaminated section of the sheet is drawn into receiving cartridge 136 and rolled onto receiving roller 124. Apparatus 100 is sufficiently lightweight and mobile to be placed across any threshold 200 in a residential, commercial, medical, science, or agricultural structure.

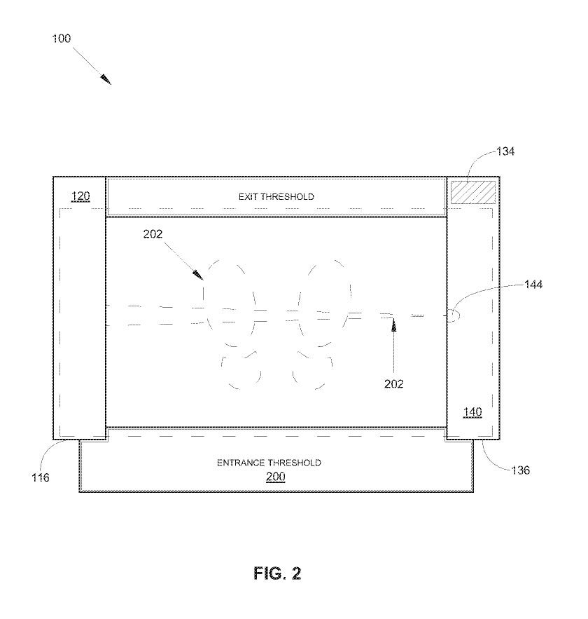

As referenced in FIG. 2, apparatus 100 includes a dispensing cartridge 116 that is configured to contain, and enable facilitated access to dispensing roller 106. Dispensing cartridge 116 is defined by a dispensing lid 118 that opens and closes to access a dispensing cavity 122. This access may be useful for replacing dispensing roller 106. Dispensing cartridge 116 is further defined by a plurality of dispensing sidewalls 120 that form a generally rectangular shape useful for retaining the cylindrically shaped dispensing roller 106. In one non-limiting embodiment, dispensing roller 106 fits longitudinally inside dispensing cartridge 116. Suitable materials for dispensing cartridge 116 may include, without limitation, stainless steel, aluminum, metal, a rigid polymer, and wood.

In some embodiments, dispensing roller 106 is defined by a dispensing outer surface 108 and an inner surface 110. Outer surface 108 of dispensing cartridge 116 rotatably carries pre-saturated sanitized sheet 102 in a rolled configuration. Pre-saturated sanitized sheet 102 is defined by a free end 104, which attaches to a receiving roller 124, discussed below. Pre-saturated sanitized sheet 102 is also unique in its disposable characteristics, such as being presoaked in a sanitizing formulation. The sanitizing formulation is configured to enable a user to wipe the soles of footwear 206 and feet for sanitizing. Sheet 102 is also sufficiently rigid to wipe off contaminants 204 and large debris, such as mud and sand.

During the process of a user wiping the footwear 206 or bare feet back and forth across the pre-saturated sanitized sheet 102, the cleaning and sanitization fluid is transferred to the soles of the footwear 206 or feet of the user, and the back and forth wiping motion results in the cleaning and sanitization of the soles. The back and forth wiping motion also facilitates the evaporation of the cleaning and sanitization fluid.

The formulation of the sanitizer that saturates sheet 102 may include readily commercially available sanitizing formulations that are currently used in numerous applications, safe, tested to kill and reduce the number of germs and viruses, and approved by the EPA and FDA, respectively. In one non-limiting embodiment, ethanol forms a substantial amount of the active ingredient, and also contain moisturizers to prevent drying of the skin. Though in other embodiments, other sanitizing formulations may be used.

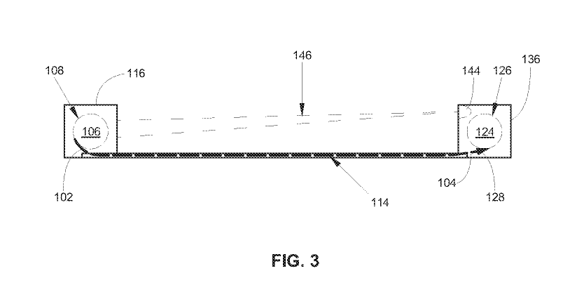

As FIG. 3 illustrates, dispensing roller 106 carries pre-saturated sanitized sheet 102 in a rolled configuration. On opposite side of threshold of the dispensing cartridge 116 and dispensing roller 106, a receiving cartridge 136 is sized and dimensioned to retain a receiving roller 124. Pre-saturated sanitized sheet 102 is stretched between dispensing roller 106 and receiving roller 124 for each respective cartridge 116, 136. Thus, the dispensing and receiving rollers 106, 124 are operatively connected by the pre-saturated sanitized sheet 102.

Sheet 102 positions above threshold 200 to enable cleaning and sanitizing of the soles of footwear 206 and feet. A fastening mechanism 128 on outer surface 126 of receiving roller 124 is configured to anchor a free end 104 of sheet 102 to receiving roller 124. Receiving roller 124 is urged to rotate, so as to draw in the contaminated sections of sheet 102 from dispensing roller 106 after footwear 206 and feet are cleaned and sanitized.

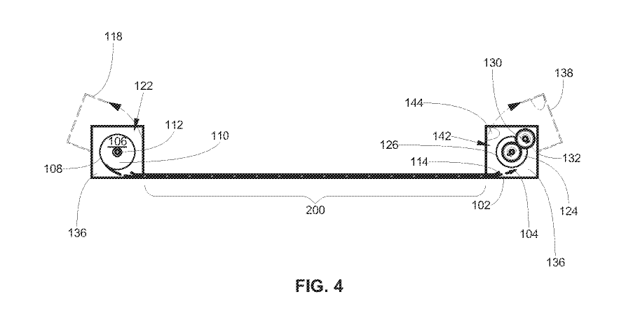

Turning now to FIG. 4, dispensing roller 106 includes a bushing 112 that is centrally disposed in the dispensing ring. Bushing 112 engages the inner surface 110 of the dispensing roller 106. Bushing 112 is arranged relative to dispensing roller 106 to cause a reduction in rotational velocity to the dispensing roller 106, as the receiving roller 124 draws the sheet 102 therefrom. This lag on the pull of the receiving roller 124 creates a taut configuration across the sheet 102. In this manner, the dispensing and receiving rollers 106, 124 create enough tension on the sheet 102, such that the sheet 102 is not displaced laterally when engaged by the footwear 206 and feet.

In some embodiments, apparatus 100 may further utilize receiving roller 124 to attach to, and receive the free end 104 of sheet 102. Receiving roller 124 is defined by an outer surface 126 having a fastening mechanism 128. Fastening mechanism 128 is configured to anchor the free end 104 of sheet 102 to receiving roller 124. Fastening mechanism 128 may include, without limitation, a slot, a plurality of hooks, a clamp, and an adhesive. This allows dispensing roller and receiving roller to attach to sheet 102 from opposite ends across the threshold 200.

In some embodiments, a receiving cartridge 136 is configured to contain and provide access to the receiving roller 124. Receiving cartridge 136 is defined by a plurality of receiving sidewalls 140 that form a generally rectangular shape effective for retaining the cylindrically shaped receiving roller 124. Receiving cartridge 136 is further defined by a receiving lid 138 that opens and closes to access a receiving cavity 142. This access may be useful for replacing the receiving roller 124. For example, FIG. 4 illustrates a sectioned side view of apparatus 100 with the lids 118, 138 for the cartridges 116, 136 open to enable access to the dispensing roller 106 and receiving roller 124 for replacement. Suitable materials for receiving cartridge 136 may include, without limitation, stainless steel, aluminum, metal, a rigid polymer, and wood.

Looking again at FIG. 4, apparatus 100 may include a gear roller 130. Gear roller 130 positions in receiving cavity 142, adjacent, and in contact with receiving roller 124. Gear roller 130 is configured to help advance sheet 102 from dispensing roller 106 to receiving roller 124, across the threshold 200. Gear roller 130 is defined by teeth 132 that operatively rotates the receiving roller 124. Gear roller teeth 132 mesh with outer surface 126 of receiving roller 124 to create rotational movement for the receiver roller in a first direction. In one non-limiting embodiment, outer surface 126 of receiving roller 124 has corresponding teeth to gear roller 130. In this manner, the rotation of receiving roller 124 in the first direction draws sheet 102 from dispensing roller 106, advancing the sheet 102 across the threshold 200 and towards receiving roller 124.

As discussed above, the frictional engagement between bushing 112 and inner surface 110 of dispensing roller 106 creates a lag on the pull from receiving roller 124. This works to retain pre-saturated sanitized sheet 102 in a generally taut configuration across the threshold 200. Specifically, the taut configuration maintains a tension across the sheet 102 in the approximate area between the rollers 106, 124 and across the threshold 200 where the user would wipe the feet or footwear 206. This helps to prevent lateral movement as a user wipes the feet during cleaning and sanitization.

Turning back to FIG. 2, a motor 134 may be used to drive the gear roller 130. Motor 134 may include an electrical motor powered by a power source, such as a rechargeable battery. Though in other embodiments, motor 134 may also be powered by an external power source. When motor 134 is actuated, the receiving roller 124 is rotatably displaced to draw sheet 102 from dispensing roller 106. In some embodiments, apparatus 100 further comprises a track 114 extending between the dispensing and receiving cartridges 116, 136. Track 114 may be disposed beneath the threshold 200. Pre-saturated sanitized sheet 102 advances across the threshold 200, generally parallel with track 114. Track 114 helps stabilize sheet 102 by providing a linear, rigid pathway for advancement.

In one non-limiting embodiment, a sensor 144 detects the absence of an object 202 between dispensing cartridge 116 and receiving cartridge 136—or across the threshold 200. Object 202 may include footwear 206, feet, lower limbs, or wheelchair components. Sensor 144 may include a motion sensor that transmits a signal 146 to the motor 134. Signal 146 is configured to command the motor 134 to power gear roller 130 for a predetermined duration, so as to draw a section of the sheet 102 from dispensing cartridge 116 to receiving cartridge 136. In essence, transmittal of signal 146 is based upon the footwear 206 or feet moving away from between the dispensing and receiving cartridge 116, 136; or after the user finishes wiping the feet. In one non-limiting embodiment, signal 146 is a radio signal.

The predetermined duration for actuating the motor 134 is sufficient to displace a contaminated section of the sheet from the threshold 200, and replace with a new section of sheet 102. In one embodiment, the duration of motor operation may be 2-6 seconds. In any case, pre-saturated sanitized sheet 102 is advanced from dispensing cartridge 116 to the receiving cartridge 136 until a clean section of the sheet 102 covers the threshold 200.

As discussed above, pre-saturated sanitized sheet 102 is disposable. Thus as shown in FIG. 4, access to the receiving cavity 142 of receiving cartridge 136 is possible through receiving lid 138. By opening receiving lid 138, it is possible to access inside the cavities 122, 142 of cartridges 116, 136 for positioning a new roll of pre-saturated sanitized sheet 102 in dispensing cartridge 116, or removing the contaminated sheet from receiving cartridge 136. Additionally, the tension across pre-saturated sanitized sheet 102 facilitates replacement from receiving cavity 142 that forms in receiving cartridge 136.

In operation, as shown in FIG. 1, a user approaches the threshold 200, wipes the soles of the footwear 206 or feet across the taut pre-saturated sanitized sheet 102, and then walks past the threshold 200 after cleaning. Sensor 144 detects motion of object 202 (footwear, feet) away from threshold 200, and automatically transmits a signal 146 to motor 134, or possible to a receiver in one of the rollers 106, 124, that commands actuation of motor 134. This causes gear roller 130 to rotate, and thereby rotate receiving roller 124; thereby drawing the sheet 102 from dispensing roller 106. Consequently, a new section of pre-saturated sanitized sheet advances over the threshold 200 for subsequent users to clean and sanitize the footwear 206 and feet.

These and other advantages of the invention will be further understood and appreciated by those skilled in the art by reference to the following written specification, claims and appended drawings.

Because many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What is claimed is:

1. An automated threshold sanitizing apparatus, the apparatus comprising:
a dispensing roller being defined by an outer surface and an inner surface, the outer surface rotatably carrying a pre-saturated sanitized sheet in a rolled configuration, the pre-saturated sanitized sheet being defined by a free end, the pre-saturated sanitized sheet being presoaked in a sanitizing formulation;
a dispensing cartridge being defined by a dispensing lid and a plurality of dispensing sidewalls forming a dispensing cavity, the dispensing cartridge housing the dispensing roller in the dispensing cavity;
a receiving roller being defined by an outer surface having a fastening mechanism, the fastening mechanism anchoring the free end of the pre-saturated sanitized sheet to the receiving roller;
a gear roller being defined by a plurality of teeth, the gear roller rotatably engaging the outer surface of the receiving roller,
whereby the teeth of the gear roller mesh with the outer surface of the receiving roller;
a motor driving the gear roller to rotate;
whereby the gear roller rotates the receiving roller,
whereby the receiving roller draws the pre-saturated sanitized sheet from the dispensing roller;
a receiving cartridge being defined by a receiving lid and a plurality of receiving sidewalls forming a receiving cavity, the receiving cartridge housing the receiving roller in the receiving cavity; and
a sensor detecting motion or presence of an object between the dispensing cartridge and the receiving cartridge, the sensor transmitting a signal based upon the motion or presence of the object, the signal actuating the motor for a predetermined duration,
whereby the predetermined duration of motor actuation causes a section of the pre-saturated sanitized sheet to be drawn from the dispensing roller,
whereby the predetermined duration of motor actuation causes the section of the pre-saturated sanitized sheet to advance from between the dispensing cartridge and the receiving cartridge on the outer surface of the receiving roller in a rolled configuration.

2. The apparatus of claim 1, further comprising a bushing, the bushing being disposed concentrically in the dispensing roller, the bushing frictionally engaging the inner surface of the dispensing roller, whereby the bushing reduces the rotational velocity of the dispensing roller.

3. The apparatus of claim 2, wherein the frictional engagement between the bushing and the inner surface of the dispensing roller helps maintain the pre-saturated sanitized sheet in a taut configuration.

4. The apparatus of claim 1, further comprising a track extending between the dispensing and receiving cartridges, wherein the pre-saturated sanitized sheet advances parallel with the track.

5. The apparatus of claim 1, wherein the receiving cartridge and the dispensing cartridge are disposed in a spaced-apart relationship separated by a threshold.

6. The apparatus of claim 5, wherein the threshold is a doormat.

7. The apparatus of claim 1, wherein the pre-saturated sanitized sheet comprises a sanitizing formulation consisting of at least ethanol.

8. The apparatus of claim 1, wherein the dispensing cartridge is generally rectangular.

9. The apparatus of claim 1, wherein the dispensing cartridge is fabricated from at least one of the following: stainless steel, aluminum, metal, a rigid polymer, and wood.

10. The apparatus of claim 1, wherein the motor comprises an electrical motor.

11. The apparatus of claim 1, wherein the fastening mechanism of the receiving roller includes at least one of the following: a slot, a plurality of hooks, a clamp, and an adhesive.

12. The apparatus of claim 1, wherein the receiving cartridge is generally rectangular.

13. The apparatus of claim 1, wherein the sensor is a motion sensor.

14. The apparatus of claim 1, wherein the signal is transmitted to the motor.

15. The apparatus of claim 1, wherein the object comprises footwear or feet.

16. The apparatus of claim 15, wherein the sensor transmits the signal based upon the footwear or feet moving away from between the dispensing and receiving cartridges.

17. An automated threshold sanitizing apparatus, the apparatus comprising:
a dispensing roller being defined by an outer surface and an inner surface, the outer surface rotatably carrying a pre-saturated sanitized sheet in a rolled configuration, the pre-saturated sanitized sheet being defined by a free end, the pre-saturated sanitized sheet being presoaked in a sanitizing formulation;
a bushing being disposed concentrically in the dispensing roller, the bushing frictionally engaging the inner surface of the dispensing roller, whereby the bushing reduces the rotational velocity of the dispensing roller;
a dispensing cartridge being defined by a dispensing lid and a plurality of dispensing sidewalls forming a dispensing cavity, the dispensing cartridge housing the dispensing roller in the dispensing cavity;
a receiving roller being defined by an outer surface having a fastening mechanism, the fastening mechanism anchoring the free end of the pre-saturated sanitized sheet to the receiving roller;
a gear roller being defined by a plurality of teeth, the gear roller rotatably engaging the outer surface of the receiving roller,
whereby the teeth of the gear roller mesh with the outer surface of the receiving roller;
a motor driving the gear roller to rotate;
whereby the gear roller rotates the receiving roller,
whereby the receiving roller draws the pre-saturated sanitized sheet from the dispensing roller,
whereby the frictional engagement between the bushing and the inner surface of the dispensing roller maintains the pre-saturated sanitized sheet in a taut configuration;
a receiving cartridge being defined by a receiving lid and a plurality of receiving sidewalls forming a receiving cavity, the receiving cartridge housing the receiving roller in the receiving cavity,
whereby the receiving cartridge and the dispensing cartridge are disposed in a spaced-apart relationship separated by a threshold; and
a motion sensor detecting motion or presence of a footwear or feet between the dispensing cartridge and the receiving cartridge, the motion sensor transmitting a signal based upon the motion or presence of the footwear or feet, the signal actuating the motor for a predetermined duration,
whereby the predetermined duration of motor actuation causes a section of the pre-saturated sanitized sheet to be drawn from the dispensing roller,
whereby the predetermined duration of motor actuation causes the section of the pre-saturated sanitized sheet to advance from between the dispensing cartridge and the receiving cartridge on the outer surface of the receiving roller in a rolled configuration.

18. The apparatus of claim 17, further comprising a track extending between the dispensing and receiving cartridges, wherein the pre-saturated sanitized sheet advances parallel with the track.

19. The apparatus of claim 17, wherein the pre-saturated sanitized sheet comprises a sanitizing formulation consisting of at least ethanol.

20. An automated threshold sanitizing apparatus, the apparatus consisting of:
a dispensing roller being defined by an outer surface and an inner surface, the outer surface rotatably carrying a pre-saturated sanitized sheet in a rolled configuration, the pre-saturated sanitized sheet being defined by a free end, the pre-saturated sanitized sheet comprising a sanitizing formulation consisting of at least ethanol;
a bushing being disposed concentrically in the dispensing roller, the bushing frictionally engaging the inner surface of the dispensing roller, whereby the bushing reduces the rotational velocity of the dispensing roller;
a dispensing cartridge being defined by a dispensing lid and a plurality of dispensing sidewalls forming a dispensing cavity, the dispensing cartridge housing the dispensing roller in the dispensing cavity;
a receiving roller being defined by an outer surface having a fastening mechanism, the fastening mechanism anchoring the free end of the pre-saturated sanitized sheet to the receiving roller;
a gear roller being defined by a plurality of teeth, the gear roller rotatably engaging the outer surface of the receiving roller,
whereby the teeth of the gear roller mesh with the outer surface of the receiving roller;
an electrical motor driving the gear roller to rotate;
whereby the gear roller rotates the receiving roller,
whereby the receiving roller draws the pre-saturated sanitized sheet from the dispensing roller,
whereby the frictional engagement between the bushing and the inner surface of the dispensing roller maintains the pre-saturated sanitized sheet in a taut configuration;
a receiving cartridge being defined by a receiving lid and a plurality of receiving sidewalls forming a receiving cavity, the receiving cartridge housing the receiving roller in the receiving cavity,
whereby the receiving cartridge and the dispensing cartridge are disposed in a spaced-apart relationship separated by a threshold;
a track extending between the dispensing and receiving cartridges,
whereby the pre-saturated sanitized sheet advances parallel with the track; and
a motion sensor detecting motion or presence of a footwear or feet between the dispensing cartridge and the receiving cartridge, the motion sensor transmitting a signal based upon the motion or presence of the footwear or feet, the signal actuating the motor for a predetermined duration,
whereby the predetermined duration of motor actuation causes a section of the pre-saturated sanitized sheet to be drawn from the dispensing roller,
whereby the predetermined duration of motor actuation causes the section of the pre-saturated sanitized sheet to advance from between the dispensing cartridge and the receiving cartridge on the outer surface of the receiving roller in a rolled configuration.
